(19) United States
(12) Patent Application Publication
(10) Pub. No.: US 2025/0177333 A1
(43) Pub. Date: Jun. 5, 2025

(54) METHOD FOR TREATING RHEUMATOID ARTHRITIS (RA) USING AN ENZYME-AND SUBSTRATE SELECTIVE EXOSITE INHIBITOR OF A DISINTEGRIN AND METALLOPROTEASE 10 (ADAM10)

(71) Applicant: Nova Southeastern University, Fort Lauderdale, FL (US)

(72) Inventor: Dmitriy Minond

(73) Assignee: Nova Southeastern University, Fort Lauderdale, FL (US)

(21) Appl. No.: 18/843,817

(22) PCT Filed: Mar. 8, 2023
(86) PCT No.: PCT/US2023/014771

§ 371 Date: Sep. 4, 2024

Related U.S. Application Data
(60) Provisional application No. 63/317,592, filed on Mar. 8, 2022.

(51) Int. Cl. A61K 31/167; A61K 31/405; A61P 19/02

(52) U.S. Cl. CPC A61K 31/167; A61K 31/405; A61P 19/02

(57) ABSTRACT

Methods for treating conditions of immune disfunction, such as rheumatoid arthritis (RA), use enzyme- and substrate-selective exosite inhibitors of A disintegrin and metalloprotease 10 (ADAM10), such as N-(3-chloro-4-methylphenyl)-2-[2-(3-methoxybenzoyl) hydrazinyl]-2-oxoacetamide (PubChem CID3117694). The method for treating a condition of immune disfunction in a subject in need thereof includes: providing a composition including a therapeutically effective dosage of a modulator of A disintegrin and metalloprotease 10 (ADAM10) and at least one acceptable pharmaceutical carrier; and administering the composition to the subject, thereby treating the condition of immune disfunction in the subject by modulating ADAM10. A composition including a therapeutically effective dosage of a modulator of A disintegrin and metalloprotease 10 (ADAM10), a therapeutically effective dosage of indomethacin, and at least one acceptable pharmaceutical carrier is for use in treating a condition of immune disfunction or inflammation in a subject in need thereof.

### STATEMENT REGARDING FEDERALLY SPONSORED RESEARCH OR DEVELOPMENT

This invention was made with government support under Grant No. DA033985 awarded by the National Institutes of Health. The U.S. government has certain rights to the invention.

### FIELD OF THE INVENTION

The invention is encompassed within the field of therapeutics for inflammatory and/or autoimmune conditions and generally relates to treatments for rheumatoid arthritis (RA), particularly relates to use of inhibitors of A disintegrin and metalloprotease 10 (ADAM10) for treatment of RA, and most particularly relates to use of N-(3-chloro-4-methylphenyl)-2-[2-(3-methoxy benzoyl) hydrazinyl]-2-oxoacetamide (PubChem CID3117694) for treatment of RA.

### BACKGROUND

A disintegrin and metaproteinase 10 (ADAM10) is member of a large group of human and nonhuman zinc-dependent enzymes (reviewed in Cerda-Costa and Gomis-Ruth. 2014). Structurally it belongs to the adamalysin family (Minond, 2020. ADAM and ADAMTS enzymes). ADAM10 is a cell surface enzyme that sheds a wide variety of cell surface proteins (Dreymueller et al., 2015; Kuhn et al., 2016; Camodeca et al., 2019; Scharfenberg et al., 2019) with importance in the progression of cancer, inflammation, and immune response, suggesting that ADAM10 can be an important target for therapy: for example, therapy of, but not limited to, rheumatoid arthritis (RA).

Rheumatoid arthritis is a common disease affecting millions in the USA alone. It is a chronic, inflammatory, and autoimmune condition that can affect the whole body and cause permanent damage to joints. There is no known cure.

30) Disease-modifying anti-rheumatic drugs (DMARDs) approved for therapy in RA include agents with the following modes of action: Janus kinase (JAK) inhibition. TNF inhibition. T cell co-stimulation blockade. IL-6 receptor inhibition. B cell depletion, and interleukin 1 inhibition. ACR70) (of DMARDs) has shown enhanced efficacy over methotrexate monotherapy (70% improvement, for responders), though the response rates are below 50%. Therefore, there is still 35 an unmet need for anti-rheumatic drugs that would improve upon ACR70 efficacy and response rate alone or in combination with approved drugs.

### SUMMARY

The invention described herein provides novel selective ADAM10 inhibitors to satisfy this unmet need for improved anti-rheumatic drugs. These novel selective ADAM10 inhibitors act via a non-Zn-binding mechanism and potentially bind outside of an active site at the hitherto unknown secondary substrate binding site (exosite).

In a basic aspect, the invention provides a new treatment approach for inflammation and/or immune disfunction. Inflammation is considered a reaction of tissue to infection or injury in which the tissue becomes swollen, red, hot, and/or painful. Immune disfunction is considered any activity or function of the immune system that is different from healthy function and produces undesired results.

In a basic aspect, the invention provides a new treatment modality for rheumatoid arthritis (RA). In another basic aspect, the invention provides novel compositions and treatments for rheumatoid arthritis.

In an aspect, the invention provides a pharmaceutical composition for treating immune disfunction, autoimmunity, inflammation, and/or rheumatoid arthritis including a therapeutically effective dosage of a modulator of A disintegrin and metalloprotease 10 (ADAM10)) and at least one acceptable pharmaceutical carrier. The “acceptable pharmaceutical carrier” can be any inactive and non-toxic agent useful for preparation of medications. The phrase “therapeutically effective dosage” or “therapeutically effective amount” refers to the amount of a composition required to achieve the desired function: for example, reduction of symptoms of inflammation, immune disfunction, and/or rheumatoid arthritis. A “modulator” can refer to any composition, compound, or substance that causes a change in the function and/or activity of another composition, compound, or substance. A non-limiting example of a modulator is an inhibitor of a protein or enzyme. A protein/enzyme inhibitor reduces and/or stops activity and/or function of the protein or enzyme. A specific, non-limiting example is an inhibitor of A disintegrin and metaproteinase 10 (ADAM10). The term “autoimmunity” refers to an immune response of an organism against its own healthy cells and/or tissues. The pharmaceutical composition can be formulated for a specific situation, for example can be formulated for oral, sublingual, buccal, nasal, rectal, parenteral, intradermal, transdermal, or intra-tracheal administration.

In another aspect, the invention provides various treatment methods for subjects in need thereof. These various methods include, but are not limited to, treating immune disfunction, treating autoimmunity, reducing inflammation, and/or treating rheumatoid arthritis. The term “subject” refers to any human or animal who will benefit from use of the compositions, methods, and/or treatments described herein. A preferred, but non-limiting subject is a human patient having rheumatoid arthritis. The method includes providing a composition including a therapeutically effective dosage of a modulator of A disintegrin and metalloprotease 10 (ADAM10) and an acceptable pharmaceutical carrier; and administering the composition to the subject. The modulator can be, but is not limited to, an inhibitor of A disintegrin and metalloprotease 10 (ADAM10). A specific, non-limiting example of such an inhibitor is N-(3-chloro-4-methylphenyl)-2-|2-(3-methoxy benzoyl) hydrazinyl|-2-oxoacetamide (PubChem CID3117694). A similar embodiment of this method includes a further step of administering an approved anti-rheumatic drug to the subject either after administering the composition or concurrently with the composition. The approved anti-rheumatic drug can be included as a component of the composition. A preferred, non-limiting, example is indomethacin.

In yet another aspect of the method, reducing inflammation and/or treating rheumatoid arthritis includes at least one of reducing swelling in tissue, reducing a disease score, and reducing an amount of at least one inflammatory biomarker. Non-limiting examples of inflammatory biomarkers are interleukin-6 (IL-6), interleukin-10 (IL-10), and C-reactive protein.

In a further aspect, the invention provides new uses for the disclosed pharmaceutical composition for treating immune disfunction, autoimmunity, inflammation, and/or rheumatoid arthritis. The composition includes a therapeutically effective dosage of a modulator of A disintegrin and metalloprotease 10 (ADAM10) and at least one acceptable pharmaceutical carrier for use in a method for treating a condition of immune disfunction or inflammation (such as RA) in a subject in need thereof. The modulator can be an inhibitor of A disintegrin and metalloprotease 10 (ADAM10). In a specific embodiment, the inhibitor of A disintegrin and metalloprotease 10 (ADAM10) is N-(3-chloro-4-methylphenyl)-2-|2-(3-methoxy benzoyl) hydrazinyl|-2-oxoacetamide (PubChem CID3117694). In another specific embodiment, the composition includes a therapeutically effective dosage of indomethacin. When the use is for RA, treating can include at least one of reducing swelling in tissue, reducing an RA disease score, and reducing an amount of at least one inflammatory biomarker. The at least one inflammatory biomarker can be at least one of interleukin-6 (IL-6), interleukin-10 (IL-10), and C-reactive protein.

Other objectives and advantages of this invention will become apparent from the following description, wherein are set forth, by way of example, certain embodiments of this invention.

### BRIEF DESCRIPTION OF THE DRAWINGS

A more complete understanding of the present invention may be obtained by references to the data shown in the accompanying drawings when considered in conjunction with the subsequent detailed description. Any embodiments illustrated in the drawings are intended only to exemplify the invention and should not be construed as limiting the invention to the illustrated embodiments.

FIG. 1 is a graph showing disease scores of rat collagen-induced arthritis model study of CID3117694. The disease score is used to assess severity of rheumatoid arthritis (RA) by measuring disease activity, functional impairment, and physical damage present.

FIG. 2 is a graph showing paw swelling measurements of rat collagen-induced arthritis model study of CID3117694.*

FIGS. 3A-F show micrographs of paw swelling observations of rat collagen-induced arthritis model study of CID3117694. In particular, the arthritis score of right hind paws of post treated groups (G1-G6) on day 28 are shown. Three joint types were observed for scoring: A: interphalangeal joint: B: metacarpophalangeal joint: C: carpal and tarsal joint. The following scale was used: Score 0: normal: Score 1: one joint type (A, B or C) has redness and swelling: Score 2: Two joint types have redness and swelling: Score 3: all three joint types have redness and swelling; and Score 4: maximal redness and swelling of the entire paw leads to disappearance of anatomical definition.

FIG. 4 is a graph showing that CID3117694 dose-dependently decreases serum levels of RA biomarker C-reactive protein.*

FIG. 5 is a graph showing that CID3117694 dose-dependently decreases serum levels of RA biomarker Interleukin-6 (IL-6).*

FIG. 6 is a graph showing that CID3117694 dose-dependently decreases serum levels of RA biomarker Interleukin-10 (IL-10).* *Data shown are mean+S.D. n=6, *P<0.05, ** P<0.01, *** P<0.001 indicates statistically significant, more significant, highly significant using a one-way ANOVA followed by Dunnett's test compared with Disease Control (CIA Ctrl).

FIG. 7 show structures of ADAM10 selective inhibitors, with hydroxamate moieties marked with circles identified with arrows.

FIGS. 8A-F show 100× light micrographs of the histopathology of collagen-induced arthritis in hind paws of rats of G6 (1-6) Group, specifically, the histopathology of right hind paws of C57BL/6 of G6 [CIA+Test Comp 50 mg Kg] post treated group. Images shown are representative H&X-stained sagittal sections of the arthritis joint as examined by light microscopy. White Arrows: Normal healthy bone & cartilage of arthritis joint: Capital H: Hyperplasia. Capital SI: Synovial inflammation.

FIGS. 9A-F show: 100× light micrographs of the histopathology of collagen-induced arthritis in hind paws of rats of G5 (1-6) Group, specifically, the histopathology of right hind paws of C57BL/6 of G5 [CIA+Test Comp 30 mg Kg] post treated group. Images shown are representative H&X-stained sagittal sections of the arthritis joint as examined by light microscopy. Capital SI: Synovial inflammation: Capital H: Hyperplasia.

FIGS. 10A-F show: 100× light micrographs of the histopathology of collagen-induced arthritis in hind paws of rats of G4 (1-6) Group, specifically, the histopathology of right hind paws of C57BL/6 of G4 [CIA+Test Comp 10 mg Kg] post treated group. Images shown are representative H&X-stained sagittal sections of the arthritis joint as examined by light microscopy. Capital SI: Synovial inflammation: Capital H: Hyperplasia: Capital P: Pannus Formation: Capital IEJ: Inflamed & eroded joint.

FIGS. 11A-F show: 100× light micrographs of the histopathology of collagen-induced arthritis in hind paws of rats of G3 (1-6) Group, specifically, the histopathology of right hind paws of C57BL/6 of G3 [CIA+Indomethacin 2.5 mg Kg] post treated group. Images shown are representative H&E-stained sagittal sections of the arthritis joint examined by light microscopy. White arrows: Normal Morphology of bone & cartilage of arthritis joint: Capital SI: Synovial inflammation: Capital H: Hyperplasia.

FIGS. 12A-F show: 100× light micrographs of the histopathology of collagen-induced arthritis in hind paws of rats of G2 (1-6) Group, specifically, the histopathology of right hind paws of C57BL/6 of G2 (CIA Control) post treated group. Images shown are representative H&X-stained sagittal sections of the arthritis joint as examined by light microscopy. Capital SI: Synovial inflammation: Capital H: Hyperplasia: Capital CE: Cartilage Erosion: Capital P: Pannus formation.

FIGS. 13A-F show: 100× light micrographs of the histopathology of collagen-induced arthritis in hind paws of rats of G1 (1-6) Group, specifically, the histopathology of right hind paws of (′57BL. 6 of G1 (Normal Saline) post treated group. Images shown are representative H&X-stained sagittal sections of the arthritis joint as examined by light microscopy. White arrows: Normal morphology of bone & cartilage of arthritis joint.

### DETAILED DESCRIPTION

For the purpose of promoting an understanding of the principles of the invention, the research will now be presented and discussed. Reference will be made to embodiments illustrated herein and specific language will be used to describe the same. It will nevertheless be understood that no limitation of the scope of the invention is thereby intended. Any alterations and further modification in the described methods, therapies, inhibitors, biomarkers, procedures, and/or compositions along with any further application of the principles of the invention as described herein, are contemplated as would normally occur to one skilled in the art to which the invention relates.

A disintegrin and metaproteinase 10 (ADAM10) is member of a large group of human and nonhuman zinc-dependent enzymes (reviewed in Cerda-Costa and Gomis-Ruth. 2014). Structurally it belongs to the adamalysin family (Minond, 2020. ADAM and ADAMTS enzymes). ADAM10 is a cell surface enzyme that sheds a wide variety of cell surface proteins (Dreymueller et al., 2015; Kuhn et al., 2016; Camodeca et al., 2019; Scharfenberg et al., 2019).

ADAM10) is comprised of several domains, namely signal sequence, prodomain, metalloproteinase domain, disintegrin domain, cysteine-rich domain, stalk region, transmembrane domain, and cytoplasmic tail (Minond, 2020), which are common for adamalysins (Takeda. 2009, 2016). ADAM10's most closely related adamalysin is ADAM17 with which it shares overall 24% amino acid sequence homology (as analyzed by Clustal Omega alignment tool). Despite low sequence homology ADAM10 and ADAM17 have a broadly overlapping and ever-growing substrate repertoire, possibly due to the lack of well-defined cleavage site primary sequence specificity (Caescu et al., 2009).

Functions of ADAM10 in any particular disease or in a normal physiological scenario are defined by the substrates that it cleaves; however, it is not well-known if ADAM10) and ADAM17 cleave the same substrates in the same setting. Therefore, inhibitors selective for ADAM10 can help differentiate its role in various scenarios.

Ability to cleave multiple substrates further complicates studies of ADAM10's role and, therefore, its validation as a target for any particular disease. ADAM10 cleaves receptors and receptor ligands such as cytokines, chemokines, cell adhesion molecules to name a few (Caescu et al., 2009; Pruessmeyer and Ludwig. 2009: Dreymueller et al., 2015: Saftig and Lichtenthaler. 2015: Moss and Minond, 2017: Wetzel et al., 2017). An ADAM10) selective inhibitor that binds to a zinc of an active site will prevent proteolysis of all ADAM10 substrates. Given that ADAM10) substrates can counteract each other's biological effect (e.g., pro- and anti-inflammatory cytokines), a substrate-specific inhibitor of ADAM10) can be useful.

This notion leads to the deeper exploration of regulatory mechanisms governing recognition and interaction between ADAM10 and ADAM17 and their substrates. These studies led to the realization that ADAM10 and ADAM17 may have multiple levels or ways of regulation of substrate recognition and processing that are outside of their active sites. Among the regulatory mechanisms known so far are trafficking of ADAMs (Lorenzen et al., 2016; Matthews et al., 2017: Seipold et al., 2018), interactions with other proteins (Koo et al., 2020). cellular membrane re-arrangement (Reiss and Bhakdi. 2017). ADAMs non-catalytic domains (Willems et al., 2010); Tape et al., 2011: Stawikowska et al., 2013: Seegar et al., 2017), topology of ADAM substrates (Stawikowska et al., 2013), enzyme (Chavaroche et al., 2014), and substrate glycosylation (Minond et al., 2012). As demonstrated by several groups these regulatory mechanisms can be targeted for a modulator discovery (Tape et al., 2011: Minond et al., 2012: Madoux et al., 2016: Seegar et al., 2017).

There has been a significant effort dedicated to the discovery of modulators of ADAM10) activity for multiple indications such as rheumatoid arthritis (RA) (Moss et al., 2008a), cancer (Moss et al., 2008b; Crawford et al., 2009; Saha et al., 2019), immune and neurodegenerative disorders (Wetzel et al., 2017). It is important to note, that for some indications (e.g., Alzheimer's disease) molecules that induce or potentiate ADAM10 activity are thought to be needed, whereas for most other indications (e.g., cancer, inflammation) the inhibitors of activity are sought after.

As shown by global knockout studies. ADAM10) (Hartmann et al., 2002) is vital for development, homeostasis and repair, which makes global inhibition of all functions of this enzyme non-feasible as a therapeutic approach. However, tissue-specific partial knockout studies of ADAM10) (Chalaris et al., 2010) demonstrated the lack of overall toxicity suggesting that local pharmacological partial inhibition of ADAM10 can be used.

The instant inventor has discovered a new class of selective ADAM10 inhibitors that act via a non-Zn-binding mechanism (Madoux et al., 2016) and potentially bind outside of an active site (FIG. 7). This non-Zn-binding mechanism of inhibition proved to be the key for ensuring selectivity of these molecules toward other Zn metalloproteinases. Additionally, the lead compound CID 3117694 from this new chemotype exhibits a unique substrate selectivity profile (Madoux et al., 2016) not observed with Zn-binding inhibitors of ADAM10, which should help avoid the off-target side effects described for Zn-binding inhibitors of ADAM10. For example, inhibition of shedding of amyloid precursor protein (APP) by ADAM10) (Fahrenholz, 2007) could lead to amyloid plaque formation in CNS. Additionally, many of Zn-binding inhibitors of metalloproteinases caused a dose limiting toxicity known as Musculo-Skeletal Syndrome (MSS) (Overall and Lopez-Otin. 2002).

Search of PubChem database for biological activity of CID3117694 revealed that it was inactive in 524 bioassays and active only against 3 targets with ADAM10 being a top target (PubChem AID 743338). Second target was hERG-CID 3117694 protected hERG from pro-arrhythmic agents (PubChem AID 1511, no EC50 value reported). Third target was DNA polymerase b (PubChem AID 485314) where CID3117694 exhibited IC50 value of 79 mM. It was inactive against adrenergic (ADRB2), muscarinic (CHRM1) and opioid receptors (OPRK1, OPRM1, and OPRD1) which are used for drug candidate safety screens (Bowes et al., 2012). These data suggest that CID 3117694 is a non-promiscuous compound which should translate into low off-target in vivo toxicity. This also suggests that inhibition of ADAM10 via a non-Zn-binding mechanism could be an effective strategy for therapy with fewer side effects due to enzyme and substrate selectivity superior to Zn-binding inhibitors.

Currently, there are no known ADAM10-targeted RA therapies on the market or in development. Thus, a need exists for novel RA therapies. Based on this need, small molecule inhibitors of ADAM10 can be an effective prevention or treatment therapy option that works via inflammatory factors that are not yet targeted by FDA-approved therapies-CD23, CX3CL1 and CXCL16. Inhibition of ADAM10 would have multiple possible benefits, decreasing (1) levels of multiple soluble inflammatory cytokines and (2) recruitment of inflammatory cells, which is an improvement over the single-target drugs (e.g., etanercept).

The main issue that likely limits study of ADAM10 inhibitors is lack of target selectivity, as it is a zinc-containing enzyme, most inhibitors bind to Zn through a “warhead”, and non-selective binding to other Zn metalloproteases by such inhibitors confers intolerable side effects.

The instant inventor has discovered a conceptually novel class of ADAM10 inhibitors acting via a non-zinc-binding inhibition mechanism. Preliminary results obtained in different cell-based models (Table 1) demonstrated efficacy of the lead compound, CID3117694, in inhibiting inflammatory cytokines and inflammatory cell recruitment and a lack of toxicity in cell-based assays.

TABLE 1
Summary of cell-based testing of the CID3117694 for
inhibition of substrates relevant in Rheumatoid arthritis
(RA). Data reported as % inhibition. Adapted from
34. PBMC-peripheral blood mononuclear cells

| | | Responsible | | [C], | % |
|---|---|---|---|---|---|
| Target | Species | ADAM | Cells | tested, μM | Inhibition |
| CXCL16 | Human | 10 | A549 | 10 | 80 |
| CCL2-induced | Human | 10 | PBMC | 10 | 50 |
| ii | | | | | |
| IL8-induced | Human | 10 | Neutro- | 10 | 100 |
| migration | | | phils | | |
| ICOSL | Mouse | 10 | B cells | 10 | 95 |

There are several selective inhibitors of ADAM10 that are available to researchers, including LT4 (ADAM10 IC50=40 nM, ADAM17 IC50=1500 nM: Zocchi et al., 2016), INCB8765 (Incyte Corporation, ADAM10 IC50=97 nM, ADAM17 IC50=2045 nM: Zhou et al., 2006), GI 254023X (Glaxo, ADAM10 IC50=5.3 nM, ADAM17 IC50=541 nM; Ludwig et al., 2005), and ADAM10 pro-domain (Biozyme Inc., ADAM10 IC50=48 nM. ADAM17 IC50)>10 mM: Moss et al., 2007). LT4. INCB8765, and GI254023X are small molecules containing hydroxamate moieties and, therefore, likely to inhibit ADAM10 via a Zn-binding mechanism (Yiotakis and Dive. 2008) (FIG. 7). ADAM10 pro-domain is a competitive inhibitor of ADAM10, but it is unknown whether it binds the active site Zn. While Zn-binding inhibitors can exhibit a degree of selectivity between closely related ADAM family members, they ultimately cannot selectively inhibit shedding of substrates. There is evidence that toxicity has been caused by off-target side effects (Dekkers et al., 1999; Newton et al., 2001: Moss and Bartsch. 2004) due to a Zn-binding mechanism of inhibition which results in broad spectrum inhibition of multiple Zn metalloproteases. Additionally. ADAM10 has been shown to cleave >70 cell surface proteins: therefore, indiscriminate inhibition of shedding of these proteins can affect multiple biological processes (reviewed in Dreymueller et al., 2015: Wetzel et al., 2017).

An ADAM10 non-Zn-binding inhibition approach overcomes drawbacks of Zn-binders. As shown by global knockout studies. ADAM10 is vital for development, homeostasis and repair, which makes global inhibition of all functions of this enzyme non-feasible as a therapeutic approach. However, tissue specific partial knockout studies of ADAM10 demonstrated the lack of overall toxicity suggesting that local pharmacological partial inhibition of ADAM10 can be used. In previous research, the instant inventor conducted a HTS campaign to identify inhibitors of ADAM10 and ADAM17. The effort identified many hits bearing prototypical Zn-binding warheads (hydroxamic acids, polycarboxylates, polyphenols, all not shown here) that, even if they showed modest selectivity for ADAM10 vs. ADAM17, would be unlikely to have wide target selectivity. This was verified by profiling studies. An exceptional HTS hit, however, is CID3117694 and related compounds (FIG. 7). While it contains highly polar functionality, these groups are not typical Zn binders, such as the hydroxamic acid groups highlighted in LT4, INCB8765, and GI254023X. CID3117694 and related compounds were micromolar-potent ADAM10 inhibitors with negligible affinity for ADAM17 and negligible affinity for a broader panel of Zn metalloproteases. This new class of selective ADAMI0 inhibitors acts via a non-Zn-binding mechanism and that potentially bind outside of an active site at the hitherto unknown secondary substrate binding site (exosite).

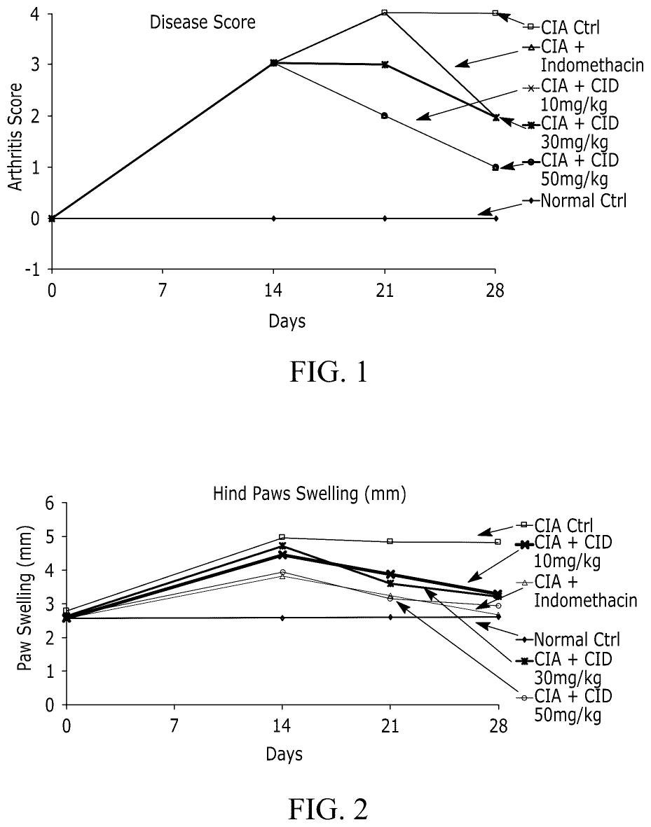

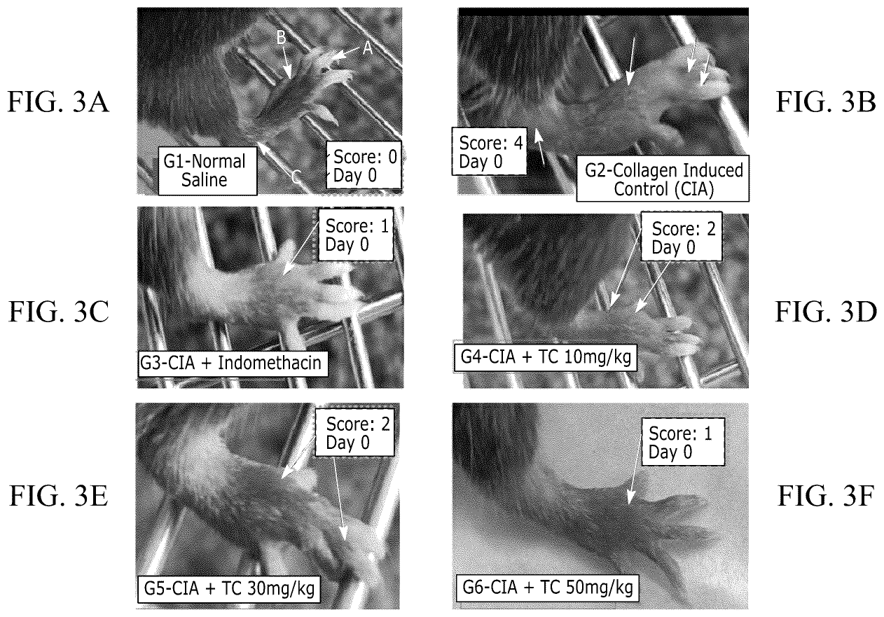

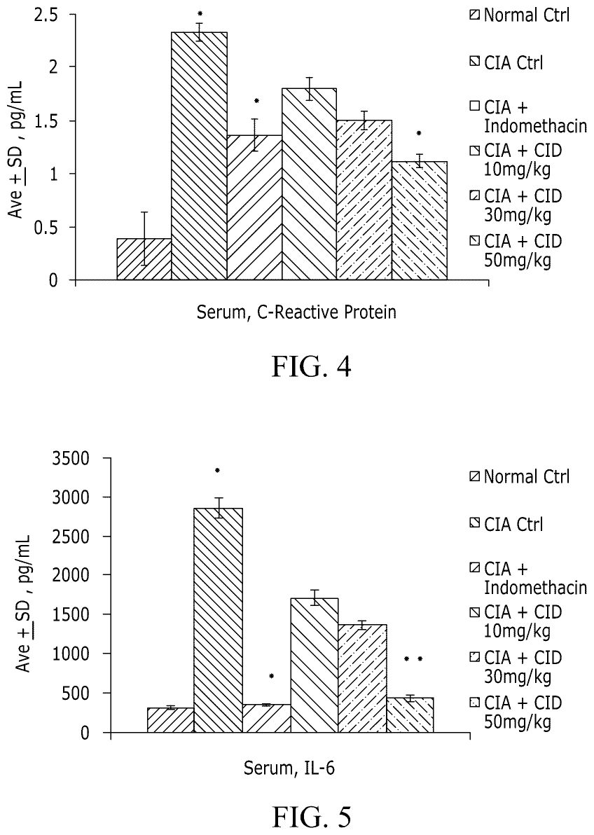

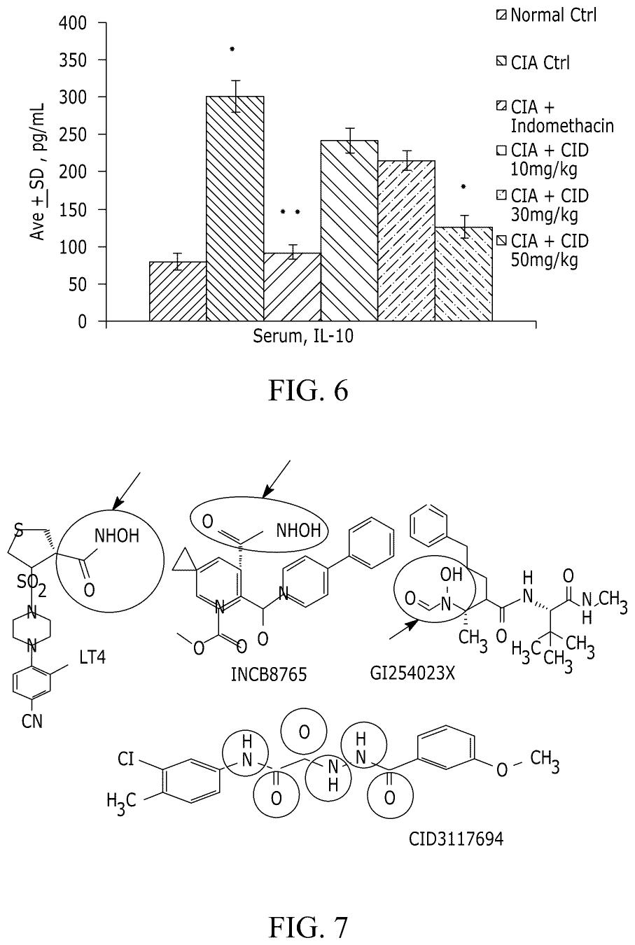

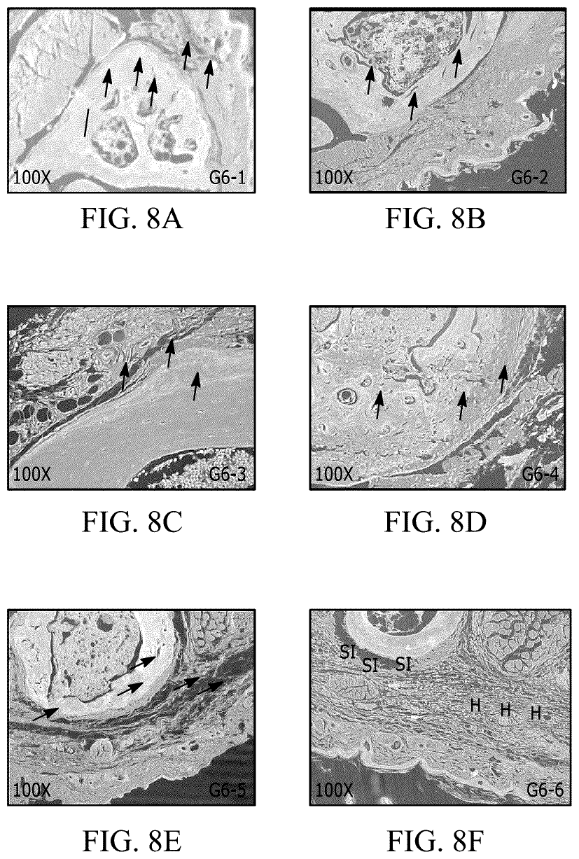

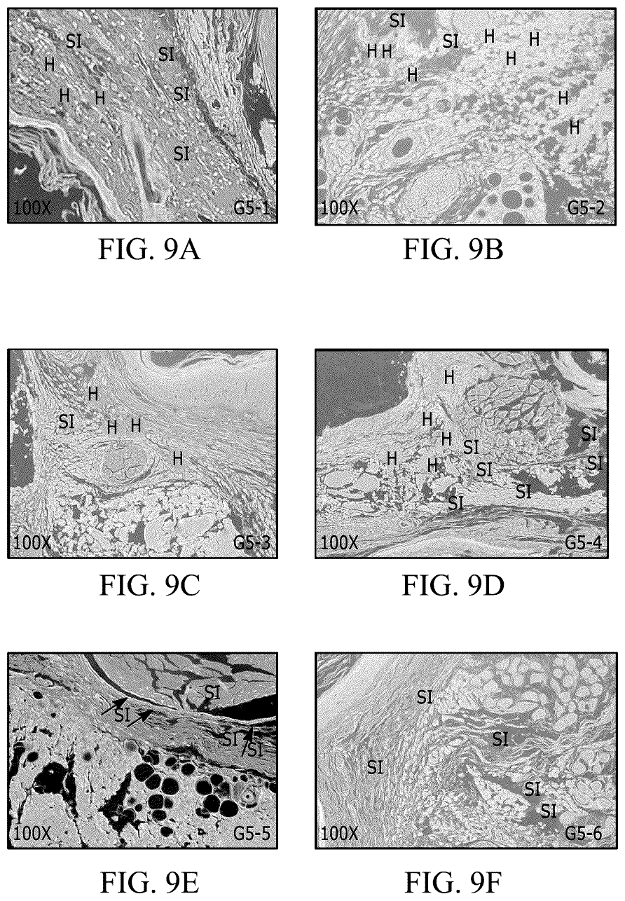

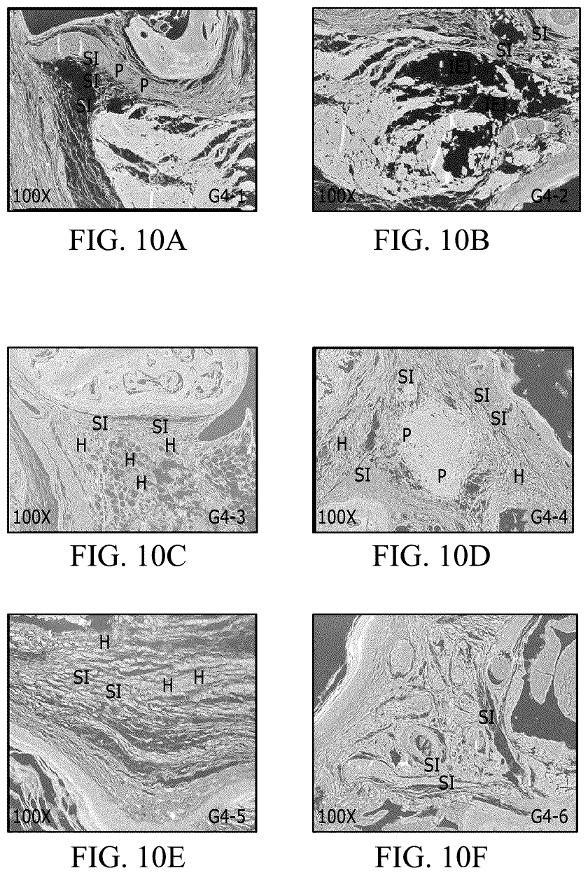

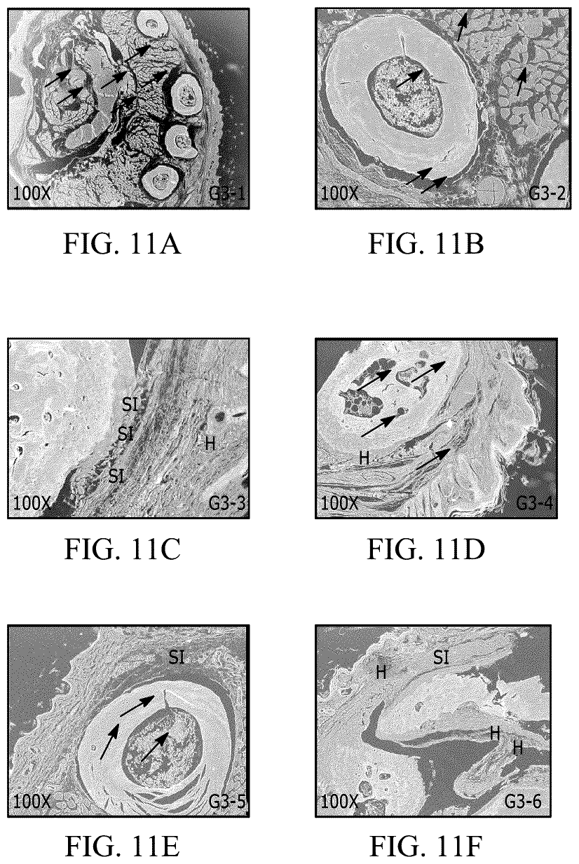

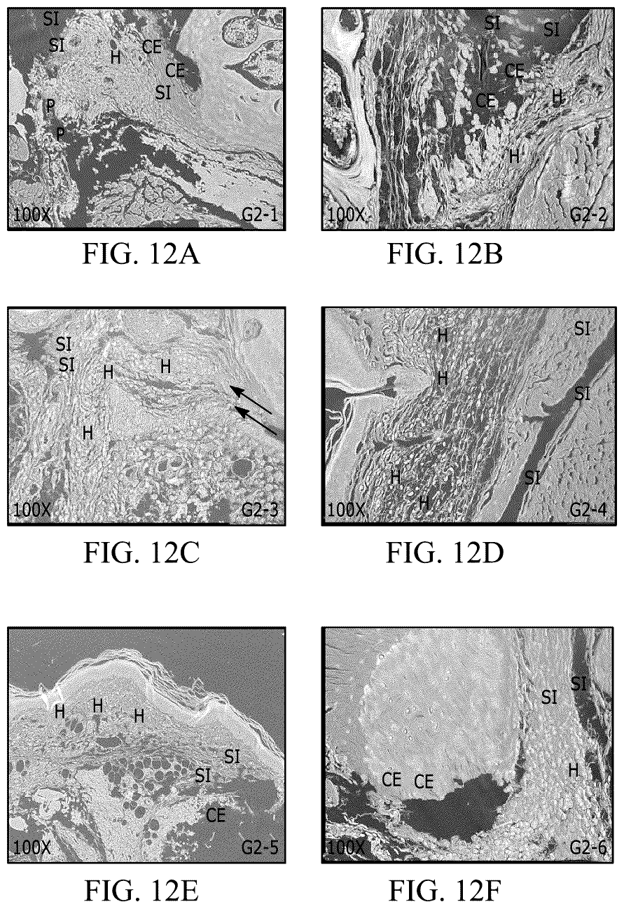

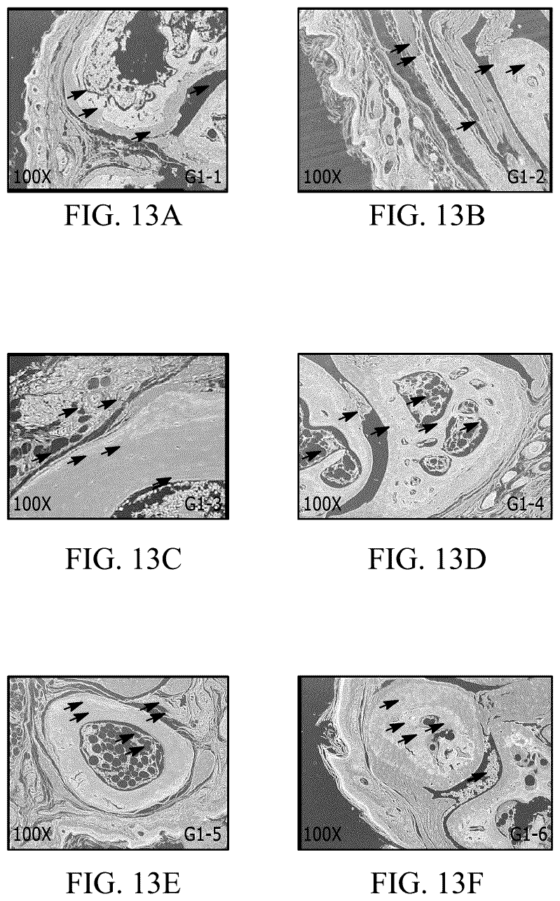

To test the efficacy of using ADAM10 inhibitor. CID3117694, for treatment of rheumatoid arthritis (RA), the instant inventor conducted a study using the mouse Collagen-induced Arthritis (CIA) in vivo model. This model is a gold standard for RA pre-clinical studies of investigational drugs. The resulting data demonstrates that CID3117694 dose-dependently lowers RA disease score (FIG. 1), paw swelling [FIG. 2 (measurement). FIGS. 3A-F (observation, photos], and inflammatory molecules (biomarkers) (FIGS. 4-6). Cytokines IL-6 and IL-10 are known ADAM10 substrates suggesting that mechanism of action of CID3117694 is based on ADAM10 activity modulation. FIGS. 8-13 are micrographs showing histopathology of the collagen-induced arthritis in the hind paws of the tested groups of rats.

### CONCLUSION

A disintegrin and metalloprotease 10 (ADAM10) is an important target for multiple therapeutics, particularly anti-rheumatic therapeutics. Overall, the current study shows that the ADAM 10 selective inhibitor, N-(3-chloro-4-methylphenyl)-2-[2-(3-methoxy benzoyl) hydrazinyl]-2-oxoacetamide (PubChem CID3117694), can overcome shortcomings of previous anti-rheumatic compounds to provide a novel treatment for rheumatoid arthritis having increased efficacy.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The methods, therapies, inhibitors, biomarkers, procedures, and/or compositions described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed, various modifications of the described modes for carrying out the invention which are obvious to those skilled in the art are intended to be within the scope of the invention.

### REFERENCES

Minond, D (2020). Novel Approaches and Challenges of Discovery of Exosite Modulators of a Disintegrin and Metalloprotease 10. Frontiers in Molecular Biosciences. 7, Article 75.
Smith, T., Tharakan, A., Martin, R. K. (2020). Targeting ADAM10 in Cancer and Autoimmunity. Frontiers in Immunology. 11, Article 499.
PubChem CID3117694 entry Pfizer and Healthline websites general information regarding rheumatoid arthritis (RA)
Bowes, J., Brown, A. J., et al. (2012). Reducing safety-related drug attrition: the use of in vitro pharmacological profiling. Nat Rev Drug Discov. 11, 909-922.
Caescu, C. I., Jeschke, G. R., and Turk, B. E. (2009). Active-site determinants of substrate recognition by the metalloproteinases TACE and ADAM10. Biochem J. 424, 79-88.
Camodeca, C., Cuffaro, D., Nuti. E., and Rossello, A. A. D. A. M. (2019). Metalloproteinases as Potential Drug Targets. Curr Med Chem. 26, 2661-2689.
Camodeca, C., Nuti, E., Tepshi, L., Boero, S., Tuccinardi, T., Stura, E. A., et al. (2016). Discovery of a new selective inhibitor of A Disintegrin And Metalloprotease 10 (ADAM-10) able to reduce the shedding of NKG2D ligands in Hodgkin's lymphoma cell models. Eur J Med Chem. 111, 193-201.
Cerda-Costa, N., and Gomis-Ruth, F. X. (2014). Architecture and function of metallopeptidase catalytic domains. Protein Sci. 23, 123-144.
Chalaris, A., Adam, N., Sina, C., Rosenstiel, P., Lehmann-Koch, J., Schirmacher, P., et al. (2010). Critical role of the disintegrin metalloprotease ADAM17 for intestinal inflammation and regeneration in mice. J Exp Med. 207, 1617-1624.
Chavaroche, A., Cudic, M., Giulianotti, M., Houghten, R. A., Fields, G. B., and Minond, D. (2014). Glycosylation of a disintegrin and metalloprotease 17 affects its activity and inhibition. Anal Biochem. 449, 68-75.
Crawford, H. C., Dempsey, P. J., Brown, G., Adam, L., and Moss, M. L. (2009). ADAM10 as a therapeutic target for cancer and inflammation. Curr Pharm Des. 15, 2288-2299.
Dekkers, P. E., Lauw, F. N., ten Hove, T., te Velde, A. A., Lumley, P., Becherer, D., et al. (1999). The effect of a metalloproteinase inhibitor (GI5402) on tumor necrosis factor-alpha (TNF-alpha) and TNF-alpha receptors during human endotoxemia. Blood. 94, 2252-2258.
Dreymueller, D., Uhlig, S., and Ludwig, A. (2015). ADAM-family metalloproteinases in lung inflammation: potential therapeutic targets. Am J Physiol Lung Cell Mol Physiol. 308, L325-L343.
Fahrenholz, F. (2007). Alpha-secretase as a therapeutic target. Curr Alzheimer Res. 4, 412-417.
Hartmann, D., de Strooper, B., Serneels, L., Craessaerts, K., Herreman, A., Annaert, W., et al. (2002). The disintegrin/metalloprotease ADAM 10 is essential for Notch signalling but not for alpha-secretase activity in fibroblasts. Hum Mol Genet. 11, 2615-2624.
Koo, C. Z., Harrison, N., Noy, P. J., Szyroka, J., Matthews, A. L., Hsia, H. E., et al. (2020). The tetraspanin Tspan15 is an essential subunit of an ADAM10 scissor complex. J Biol Chem
Kuhn, P. H., Colombo, A. V., Schusser, B., Dreymueller, D., Wetzel, S., Schepers, U., et al. (2016). Systematic substrate identification indicates a central role for the metalloprotease ADAM10 in axon targeting and synapse function. Elife 5, e012748.
Lorenzen, I., Lokau, J., Korpys, Y., Oldefest, M., Flynn, C. M., Kunzel. U., et al. (2016). Control of ADAM17 activity by regulation of its cellular localisation. Sci Rep. 6, 35067.
Ludwig, A., Hundhausen, C., Lambert, M. H., Broadway, N., Andrews, R. C., Bickett, D. M., et al. (2005). Metalloproteinase inhibitors for the disintegrin-like metalloproteinases ADAM10 and ADAM17 that differentially block constitutive and phorbol ester-inducible shedding of cell surface molecules. Comb Chem High Throughput Screen. 8, 161-171.
Madoux, F., Dreymuller, D., Pettiloud, J. P., Santos, R., Becker-Pauly, C., Ludwig, A., et al. (2016). Discovery of an enzyme and substrate selective inhibitor of ADAM10 using an exosite-binding glycosylated substrate. Sci Rep. 6, 11.
Matthews, A. L., Noy, P. J., Reyat, J. S., and Tomlinson, M. G. (2017). Regulation of A disintegrin and metalloproteinase (ADAM) family sheddases ADAM10 and ADAM17: The emerging role of tetraspanins and rhomboids. Platelets. 28, 333-341.
Minond, D., Cudic, M., Bionda, N., Giulianotti, M., Maida, L., Houghten, R. A., et al. (2012). Discovery of novel inhibitors of a disintegrin and metalloprotease 17 (ADAM17) using glycosy lated and non-glycosylated substrates. J Biol Chem. 287, 36473-36487.
Moss, M. L., and Bartsch, J. W. (2004). Therapeutic benefits from targeting of ADAM family members. Biochemistry. 43, 7227-7235.
Moss, M. L., and Minond, D. (2017). Recent Advances in ADAM17 Research: A Promising Target for Cancer and Inflammation. Mediators Inflamm. 2017, 9673537.
Moss, M. L., Bomar, M., Liu, Q., Sage, H., Dempsey, P., Lenhart, P. M., et al. (2007). The ADAM10 prodomain is a specific inhibitor of ADAM10 proteolytic activity and inhibits cellular shedding events. J Biol Chem. 282, 35712-35721.
Moss, M. L., Sklair-Tavron, L., and Nudelman, R. (2008). Drug insight: tumor necrosis factor-converting enzyme as a pharmaceutical target for rheumatoid arthritis. Nat Clin Pract Rheumatol. 4, 300-309.
Moss, M. L., Stoeck, A., Yan, W., and Dempsey, P. J. (2008b). ADAM10 as a target for anti-cancer therapy. Curr Pharm Biotechnol. 9, 2-8.
Newton, R. C., Solomon, K. A., Covington, M. B., Decicco, C. P., Haley, P. J., Friedman, S. M., et al. (2001). Biology of TACE inhibition. Annals of the rheumatic diseases. 60 (Suppl. 3), iii25-iii32.
Overall, C. M., and Lopez-Otin, C. (2002). Strategies forMMPinhibition in cancer: innovations for the post-trial era. Nat Rev Cancer. 2, 657-672.
Pruessmeyer, J., and Ludwig, A. (2009). The good, the bad and the ugly substrates for ADAM10 and ADAM17 in brain pathology, inflammation and cancer. Semin Cell Dev Biol. 20, 164-174.
Reiss, K., and Bhakdi, S. (2017). The plasma membrane: Penultimate regulator of ADAM sheddase function. Biochim Biophys Acta Mol Cell Res. 1864 (11 Pt B). 2082-2087.
Saftig, P., and Lichtenthaler, S. F. (2015). The alpha secretase ADAM10: A metalloprotease with multiple functions in the brain. Prog Neurobiol. 135, 1-20.
Saha, N., Robev, D., Himanen, J. P., and Nikolov, D. B. (2019). ADAM proteases: Emerging role and targeting of the non-catalytic domains. Cancer Lett. 467, 50-57.
Scharfenberg, F., Helbig, A., Sammel, M., Benzel, J., Schlomann, U., Peters, F., et al. (2019). Degradome of soluble ADAM10 and ADAM17 metalloproteases. Cell Mol Life Sci. 77, 331-350.
Seegar, T. C. M., Killingsworth, L. B., Saha, N., Meyer, P. A., Patra, D., Zimmerman, B., et al. (2017). Structural Basis for Regulated Proteolysis by the alpha-Secretase ADAM10. Cell 171, 1638-48e7.
Seipold, L., Altmeppen, H., Koudelka, T., Tholey, A., Kasparek, P., Sedlacek, R., et al. (2018). In vivo regulation of the A disintegrin and metalloproteinase 10 (ADAM10) by the tetraspanin 15. Cell Mol Life Sci. 75, 3251-3267.
Stawikowska, R., Cudic, M., Giulianotti, M., Houghten, R. A., Fields, G. B., and Minond, D. (2013). Activity of ADAM17 (a disintegrin and metalloprotease 17) is regulated by its noncatalytic domains and secondary structure of its substrates. J Biol Chem. 288, 22871-22879.
Takeda, S. (2009). Three-dimensional domain architecture of the ADAM family proteinases. Semin Cell Dev Biol. 20, 146-152.
Tape, C. J., Willems, S. H., Dombernowsky, S. L., Stanley, P. L., Fogarasi, M., Ouwehand, W., et al. (2011). Cross-domain inhibition of TACE ectodomain. Proc Natl Acad Sci USA. 108, 5578-5583.
Wetzel, S., Seipold, L., and Saftig, P. (2017). The metalloproteinase ADAM10: A useful therapeutic target? Biochim Biophys Acta Mol Cell Res. 1864 (11 Pt B), 2071-2081.
Willems, S. H., Tape, C. J., Stanley, P. L., Taylor, N. A., Mills, I. G., Neal, D. E., et al. (2010). Thiol isomerases negatively regulate the cellular shedding activity of ADAM17. Biochem J. 428, 439-450.
Yiotakis, A., and Dive, V. (2008). Synthetic active site-directed inhibitors of metzincins: achievement and perspectives. Mol Aspects Med. 29, 329-338.
Zhou, B. B., Peyton, M., He, B., Liu, C., Girard, L., Caudler, E., et al. (2006). Targeting ADAM-mediated ligand cleavage to inhibit HER3 and EGFR pathways in non-small cell lung cancer. Cancer Cell. 10, 39-50.
Zocchi, M. R., Camodeca, C., Nuti, E., Rossello, A., Vene, R., Tosetti, F., et al. (2016). ADAM10 new selective inhibitors reduce NKG2D ligand release sensitizing Hodgkin lymphoma cells to NKG2D-mediated killing. Oncoimmunology. 5, e1123367.

1-46. (canceled)

47. A method for treating a condition of immune disfunction or inflammation in a subject in need thereof, the method comprising:
providing a composition including a therapeutically effective dosage of a modulator of A disintegrin and metalloprotease 10 (ADAM-10) and at least one acceptable pharmaceutical carrier; and
administering the composition to the subject, thereby treating the condition of immune disfunction or inflammation in the subject by modulating ADAM-10.

48. The method according to claim 47, wherein the modulator is an inhibitor of A disintegrin and metalloprotease 10 (ADAM-10).

49. The method according to claim 48, wherein the inhibitor of A disintegrin and metalloprotease 10 (ADAM-10) is N-(3-chloro-4-methylphenyl)-2-[2-(3-methoxybenzoyl) hydrazinyl]-2-oxoacetamide (PubChem CID3117694).

50. The method according to claim 48, wherein the condition of immune disfunction or inflammation is rheumatoid arthritis (RA).

51. The method according to claim 50, further comprising administering a therapeutically effective dosage of indomethacin to the subject,
wherein the administering includes at least one of administering the therapeutically effective dosage of indomethacin prior to administering the composition, administering the therapeutically effective dosage of indomethacin concurrently with the composition, and administering the therapeutically effective dosage of indomethacin as part of the composition.

52. The method according to claim 48, wherein the inflammation is a result of rheumatoid arthritis (RA).

53. The method according to claim 52, wherein treating the inflammation includes at least one of reducing swelling in tissue, reducing an RA disease score, and reducing an amount of at least one inflammatory biomarker.

54. The method according to claim 53, wherein the at least one inflammatory biomarker is at least one of interleukin-6 (IL-6), interleukin-10 (IL-10), and C-reactive protein.

55. A method for treating rheumatoid arthritis (RA) in a subject in need thereof, the method comprising:
providing a composition including a therapeutically effective dosage of a modulator of A disintegrin and metalloprotease 10 (ADAM-10) and at least one acceptable pharmaceutical carrier; and
administering the composition to the subject, thereby treating the rheumatoid arthritis (RA) in the subject by modulating ADAM-10.

56. The method according to claim 55, wherein the modulator is an inhibitor of A disintegrin and metalloprotease 10 (ADAM-10).

57. The method according to claim 56, wherein the inhibitor of A disintegrin and metalloprotease 10 (ADAM-10) is N-(3-chloro-4-methylphenyl)-2-[2-(3-methoxybenzoyl) hydrazinyl]-2-oxoacetamide (PubChem CID3117694).

58. The method according to claim 56, wherein treating the rheumatoid arthritis (RA) includes at least one of reducing swelling in tissue, reducing an RA disease score, and reducing an amount of at least one inflammatory biomarker.

59. The method according to claim 58, wherein the at least one inflammatory biomarker is at least one of interleukin-6 (IL-6), interleukin-10 (IL-10), and C-reactive protein.

60. The method according to claim 56, further comprising administering a therapeutically effective dosage of indomethacin to the subject,
wherein the administering includes at least one of administering the therapeutically effective dosage of indomethacin prior to administering the composition, administering the therapeutically effective dosage of indomethacin concurrently with the composition, and administering the therapeutically effective dosage of indomethacin as part of the composition.

61. A method for treating rheumatoid arthritis (RA) in a subject in need thereof, the method comprising:
providing a composition including a therapeutically effective dosage of N-(3-chloro-4-methylphenyl)-2-[2-(3-methoxybenzoyl) hydrazinyl]-2-oxoacetamide (PubChem CID3117694) and at least one acceptable pharmaceutical carrier; and
administering the composition to the subject, thereby treating the rheumatoid arthritis (RA) in the subject.

62. The method according to claim 61, wherein treating the rheumatoid arthritis (RA) includes at least one of reducing swelling in tissue, reducing an RA disease score, and reducing an amount of at least one inflammatory biomarker.

63. The method according to claim 62, wherein the at least one inflammatory biomarker is at least one of interleukin-6 (IL-6), interleukin-10 (IL-10), and C-reactive protein.

64. The method according to claim 61, further comprising administering a therapeutically effective dosage of indomethacin to the subject,
wherein the administering includes at least one of administering the therapeutically effective dosage of indomethacin prior to administering the composition, administering the therapeutically effective dosage of indomethacin concurrently with the composition, and administering the therapeutically effective dosage of indomethacin as part of the composition.
